(19) United States
(12) Patent Application Publication
(10) Pub. No.: US 2003/0069482 A1
(43) Pub. Date: Apr. 10, 2003

(54) Sampling article for determining quantitative and qualitative drug transfer to skin

(76) Inventors: Jerome James Workman JR., Appleton, WI (US); Corey Thomas Cunningham, Larsen, WI (US); Terry William Adler, Neenah, WI (US)

Correspondence Address: Pauley Petersen Kinne & Fejer, Suite 365, 2800 W. Higgins Road, Hoffman Estates, IL, 60195, US

(21) Appl. No.: 09/973,680

(22) Filed: Oct. 9, 2001

(51) Int. Cl. A61B 5/00; A61B 5/05

(52) U.S. Cl. 600/306; 600/345

(57) ABSTRACT

An article for measuring external drug transfer to skin surfaces includes a high molecular weight polyolefin substrate film and a skin-adhering element attached to the polyolefin substrate film. The substrate film has a texture that simulates skin roughness and topography on the surface facing a drug delivery medium. The substrate film, skin-adhering element, and all other components of, or in contact with, the sampling article provide minimum interference with chemical analysis of the active drug being measured.

### BACKGROUND OF THE INVENTION

Drug transfer studies are carried out to measure drug transfer to skin surfaces from drug-containing media such as drug transfer patches, diapers, wound dressings, facial tissues, ointments, pastes, lotions, and other bandages and wipes. In order to obtain accurate results, it is important that any device used to take the sample of the drug not interfere with the activity of, or chemical analysis of, the drug itself.

Devices conventionally used to take such drug samples often include film substrates, adhesives, and/or packaging that interferes with chemical analysis of the active drug contained in or on the drug transfer media. More particularly, these devices often include chemical groups that interfere with either direct analysis or extraction analysis techniques where ultraviolet (UV), visible (Vis), or infrared (IR) spectroscopy; gas chromatography (GC); gas chromatography-mass spectrometry (GC-MS); mass spectrometry (MS); liquid chromatography (LC); liquid chromatography-mass spectrometry (LC-MS); or mass spectrometry/mass spectrometry (MS-MS) are used.

Furthermore, conventional drug-sampling devices are often of a shape that is convenient to manufacture, rather than a shape that is optimized for use in sampling drugs. Drug-sampling devices that are not optimally shaped for their intended use may be too bulky or too awkwardly-shaped to accurately sample the drug being tested. In addition, conventional drug-sampling devices typically have a texture that is different than human skin, which results in inaccurate quantitative drug transfer data.

Drug transfer studies must generally be completed in large numbers. Drug-sampling devices that interfere with, or inaccurately quantify, the drug being tested result in skewed data, thus rendering such studies useless.

There is a need or desire for a drug-sampling device that emulates the surface of human skin, while being constructed of materials which do not interfere with a variety of analytical chemical techniques.

### SUMMARY OF THE INVENTION

In response to the discussed difficulties and problems encountered in the prior art a new sampling article has been discovered. The present invention is directed to a sampling article for quantitatively and qualitatively measuring drug transfer to skin surfaces from drug-containing media. The sampling article is specifically designed to emulate the surface of the human skin, while being constructed of materials that provide minimum (i.e., little or no) interference with chemical analysis of the active drug contained in or on the drug-containing media.

The sampling article is placed directly onto a skin surface in a location and manner representative of the actual use conditions of the drug-containing media. On the surface facing the drug-containing media, the article has a texture that simulates the roughness and topography of human skin. The article adheres to the skin surface and acts as a substrate for sampling drug transfer to the skin.

None of the materials used for construction of the sampling article contain chemicals that interfere with the active drug components contained within the drug formulation being tested. Specifically, none of the materials used for construction of the article or in contact with the article as packaging contain any derivative or compound containing the active drug, a direct interferent of that drug, or compounds and functional groups which completely overlap or obscure the analysis of the drug using conventional analytical techniques. More specifically, the article and its packaging do not contain aromatic organic compounds, polyenes, acrylates, esters, waxes, dimethicones or silicone-based compounds, which interfere with common drug components.

The shape and structure of the article is suitably designed to provide optimal fit to the body area of interest. The specific size may vary, depending upon the specific area of the body and application required.

With the foregoing in mind, it is a feature and advantage of the invention to provide a sampling device for obtaining and measuring drug transfer samples, which emulates the surface of human skin and does not interfere with a variety of conventional analytical chemical techniques.

### BRIEF DESCRIPTION OF THE DRAWINGS

FIG. 1 is a side view of a sampling article;

FIG. 2 is a front view of packaging material encasing a sampling article;

FIG. 3 is a perspective view of a sampling article applied to a finger;

FIG. 4 is a perspective view of a sampling article applied below a nose; and

FIG. 5 is a perspective view of a sampling article applied to a baby's buttocks.

### DEFINITIONS

Within the context of this specification, each term or phrase below will include the following meaning or meanings.

“Drug,” as used herein, refers to any medicant, including anti-inflammatories, anesthetics, analgesics, anti-bacterial treatments, as well as vitamins, nutrients, and any other treatment used to improve a person's health.

“Drug-containing medium” refers to a vehicle used to deliver a drug to human skin.

“External drug transfer” refers to the application of a drug to human skin, as opposed to a drug that is ingested.

“Human skin topography” refers to a surface having contours, such as lumps, depressions, and lines, modeled after the contours present on human skin.

“Non-interfering” refers to a chemical that, in the presence of a subject chemical, does not react with the subject chemical nor affect the results of chemical analysis techniques performed on the subject chemical.

“Packaging materials” refer to any type of container, wrapping, or shipping materials used to encase or pack an item for purposes of shipment, storage, or marketing, for example.

“Skin-adhering” is a term used herein to describe an element, surface, or the like, that is capable of adhering to the skin.

These terms may be defined with additional language in the remaining portions of the specification.

### DETAILED DESCRIPTION OF THE PRESENTLY PREFERRED EMBODIMENTS

The invention is directed to a sampling article for quantitatively and qualitatively measuring external drug transfer to skin surfaces from drug-containing media. The principles of the present invention can be applied to the sampling of externally transferred drugs transferred by any suitable drug-containing media, including but not limited to drug transfer patches, diapers, wound dressings, facial tissues, ointments, pastes, lotions, and other bandages and wipes.

The sampling article of the invention is designed to simulate human skin for purposes of measuring drug transfer to skin from drug-containing media. After the drug is transferred to the sampling article, the sampling article can be tested to determine, quantitatively, how much of the drug was transferred, and, qualitatively, exactly what was transferred. In order to achieve accurate results, it is important that the sampling article is constructed of materials that provide no interference, or at least minimal interference, with chemical analysis of the active drug components contained in or on the drug transfer media.

Thus, the sampling article must not contain chemical groups, and must not be in contact with any materials prior to use that contain chemical groups, which interfere with either direct analysis or extraction analysis techniques where ultraviolet (UV), visible (Vis), or infrared (IR) spectroscopy; gas chromatography (GC); gas chromatography-mass spectrometry (GC-MS); mass spectrometry (MS); liquid chromatography (LC); liquid chromatography-mass spectrometry (LC-MS); or mass spectrometry/mass spectrometry (MS-MS) are used.

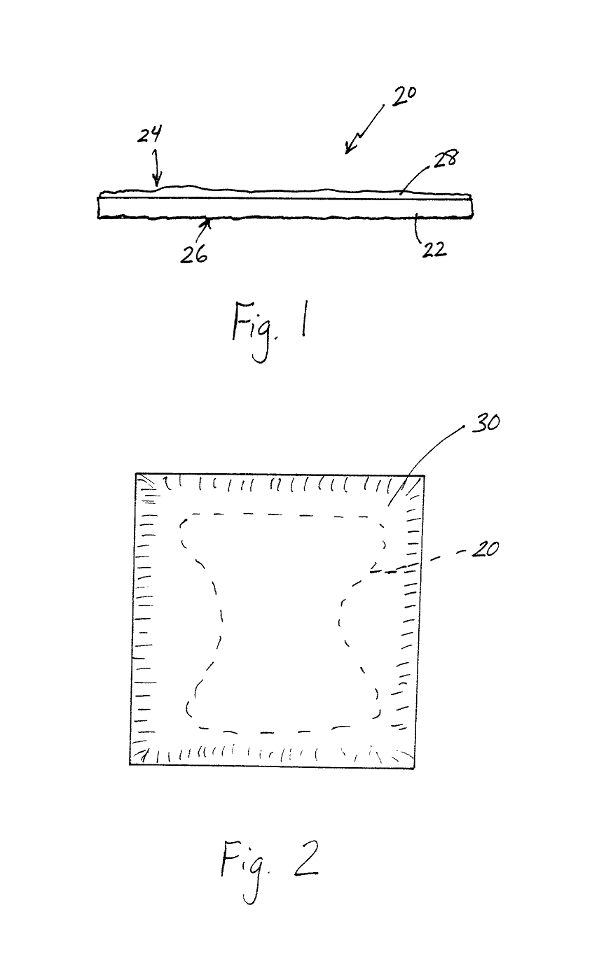

FIG. 1 illustrates a side view of a sampling article 20 of the invention. The sampling article 20 includes a substrate 22 having a skin-adhering surface 24 and a skin-emulating surface 26. The substrate 22 is suitably a film made from polyolefin materials, such as polyethylene, polypropylene, or polytetrafluoroethylene. These materials have a high weight-average molecular weight of about 3 to about 6 kilograms/mole, suitably about 3 to about 4 kilograms/mole for the polyolefin-based materials. For the polytetrafluoroethylene, having a high weight-average molecular weight of about 35 to about 65 kilograms/mole, suitably about 45 to about 55 kilograms/mole.

In addition, other suitable substrate materials include metallic foil films or metallized films, including but not restricted to gold, aluminum, copper, tin, magnesium, silver, iron, zinc, and platinum. However, safety and cost constraints indicate that foil films or metallized films of aluminum are not necessarily ideal. These materials also exhibit the useful property of malleability.

Other polymer films which may be used as the substrate 22 and which may be useful in reducing analytical interferences to chemical analysis of transferred drug components include, but are not restricted to, poly(methyl methacrylate), poly(vinyl alcohol), poly(ethylene oxide), poly(ethylene terephthalate), polycaprolactam, poly(hexamethylene adipamide), poly(α-1,6-D-glucose), polydimethylsiloxanes, and poly(cis-1,4-isoprene). Each of these polymers made into films may prove useful for specific analytical requirements, but they do not represent a universal solution to prevention of analytical interferences as provided by the metallic foil films, metallized films, or polymer films of the polytetrafluoroethylene family.

The substrate 22 should also have a texture such that human skin roughness and human skin topography are simulated on the skin-emulating surface 26. More particularly, the skin-emulating surface 26 of the substrate 22 has a roughness of about 19 to about 32 microns, suitably about 25 microns average surface roughness, measured by using a Taylor-Hobson S5 contact profilometer configured with a 2-micron radius tip and laser interferometer pickup. The average roughness is reported as the arithmetic sum of all deviations about the best-fit mean plane through the topographical surface data. The substrate may have a thickness of about 10 to about 2500 microns, suitably about 50 to about 250 microns; and an area of about 4 to about 25 square centimeters, suitably about 2.25 to about 6.25 square centimeters.

The skin-adhering surface 24 of the sampling article 20 includes a skin-adhering element 28 designed to adhere the article 20 to a wearer's skin. The skin-adhering element 28 may include, for example, an adhesive formula, a statically adhesive material, or a gel adhesive. The skin-adhering element 28 used to directly attach the article substrate 22 to the skin must not interfere with the analysis of the drug when using any of the aforementioned analytical techniques. Specifically, the adhesive system within the solvent must have all components with boiling points in excess of 250° Celsius, and minimum molecular weights greater than 1,500 daltons. Basic useful adhesive chemistry has been described broadly in the patent art related to the term “pressure sensitive adhesives.” However, it is essential to note that the exception to all adhesive formulas in this invention is that the absolute lower molecular weight limit for chemical components of the adhesive systems must exceed 1,500 daltons, with no upper molecular weight limit. These chemical components of the adhesive system must also have boiling points in excess of 250° Celsius. The basic formulations taught in the prior art have been modified in the following text to include the key issue of this invention related to adhesives, that being the specific minimum allowable molecular weight of the individual chemical components.

Examples of suitable adhesive formulas include hydrogel adhesives made of a water-soluble long chain greater than 1,500 daltons (meth)acrylate ester monomer; a hydrogel adhesive made of a co-polymer formed by copolymerizing a first water-soluble long chain (meth)acrylate ester monomer with a second water-soluble monomer with all components to exceed a minimum molecular weight of 1,500 daltons, as described in U.S. Pat. No. 5,674,275 and European Patent No. 0 676 457 A1 entitled POLYACRYLATE AND POLYMETHACRYLATE ESTER BASED HYDROGEL ADHESIVES, both of which are hereby incorporated by reference.

A more specific example of a suitable adhesive formula is a latex pressure sensitive adhesive including: (a) a copolymer mixture comprising about 40 to about 70 weight percent of a solid phase, the solid phase comprising the reaction product of: (i) about 70 to about 98.5 percent by weight of monomer selected from the group consisting of C to C alkyl acrylate ester monomer and mixtures thereof; (ii) about 0 to about 20 percent by weight of monomer selected from the group consisting of vinyl esters, C to C esters of (meth)acrylic acid, styrene, and mixtures thereof; (iii) about 1 to about 10 percent by weight of polar monomer copolymerizable with said monomer of element (a)(i) and element (a)(ii); (iv) about 0.5 to about 20 percent by weight of a hydrophobic polymer which is incapable of reaction with said monomers of elements (a)(i), (a)(ii), and (a)(iii), wherein said hydrophobic polymer has molecular weight ranging from about 1,500 to about 50,000 daltons; (v) about 0.01 to about 1 percent by weight of an initiator; (vi) about 1 to about 10 percent by weight of an ionic copolymerizable surfactant; (vii) about 0 to 1 percent by weight of a chain transfer agent; and (viii) about 0 to 5 percent by weight of a crosslinking agent; wherein the percentages of (v), (vi), (vii), and (viii) are each based on the total weight of (i) plus (ii) plus (iii) plus (iv) and wherein the percentages of (i), (ii), (iii), and (iv) are each based on the total weight of (i) plus (ii) plus (iii) plus (iv); and (b) about 30 to about 60 percent by weight of an aqueous phase; wherein said weight percentages of (a) and (b) are each based on the total weight of said latex, as described in European Patent No. 0 554 832 B1 entitled HIGH SOLIDS MOISTURE RESISTANT LATEX PRESSURE-SENSITIVE ADHESIVE, hereby incorporated by reference.

Another adhesive system composed of polyalkyloxazolines of molecular weight within a range from about 1,500 to about 2,000,000 daltons could be used. Polymers of molecular weight below 1,500 provide only weak reinforcement, and those above 2,000,000 produce pressure sensitive adhesives which exhibit too large a drop in peel adhesion and which are not readily adaptable to hot melt coating. Molecular weights of from about 2,000 to about 500,000 are preferred, with from about 5,000 to about 50,000 being most preferred. Also, preferred are oxazo-line polymers where x is 1, R is hydrogen, and R1 from hydrogen and alkyl groups containing up to about 10 carbon atoms, with the most preferred R substituents being hydrogen, methyl, ethyl, and propyl, as described in U.S. Pat. No. 4,737,410 entitled POLYALKYLOXAZOLINE-REINFORCED ACRYLIC PRESSURE-SENSITIVE ADHESIVE COMPOSITION, which is hereby incorporated by reference.

In addition, a means of electrostatic adhesion can be used when the polymer substrate is less than 80 microns in thickness. Electrostatic charge will cause the polymer film to adhere to the skin surface. This is not particularly useful when significant abrasion forces are applied to the polymer surface when in place, but there are certain specific cases of use when electrostatic adhesion may be optimum. These cases include the spraying of drug-containing medium onto a solid surface using an aerosol device, or when a drug material is allowed to free flow onto a solid surface. The elimination of the adhesive simplifies the analysis even further than the use of high molecular weight component containing adhesives.

Packaging materials in contact with the substrate 22 must not defeat the purposes of the invention. Because the packaging materials are in contact with the substrate 22 which comes into contact with the drug being measured, the packaging materials should be non-interfering with respect to analysis of the drug being measured. As mentioned, all materials used for construction of the article 20 or in contact with the article 20 as packaging must not contain any derivative or compound containing the active drug, a direct interferent of that drug, or compounds and functional groups which completely overlap or obscure the analysis of the drug using the aforementioned analytical techniques. Specifically, neither the sampling article 20 nor its packaging should contain aromatic organic compounds, polyenes, acrylates, esters, waxes, dimethicones or silicone-based compounds. Furthermore, all materials used for the sampling article 20 and its packaging should have melting points in excess of about 250 degrees Celsius, and minimum molecular weights greater than about 1,500 grams/mole, because this molecular weight prevents interference when using chromatographic and mass spectrometry analytical methods for analysis of typical drug components.

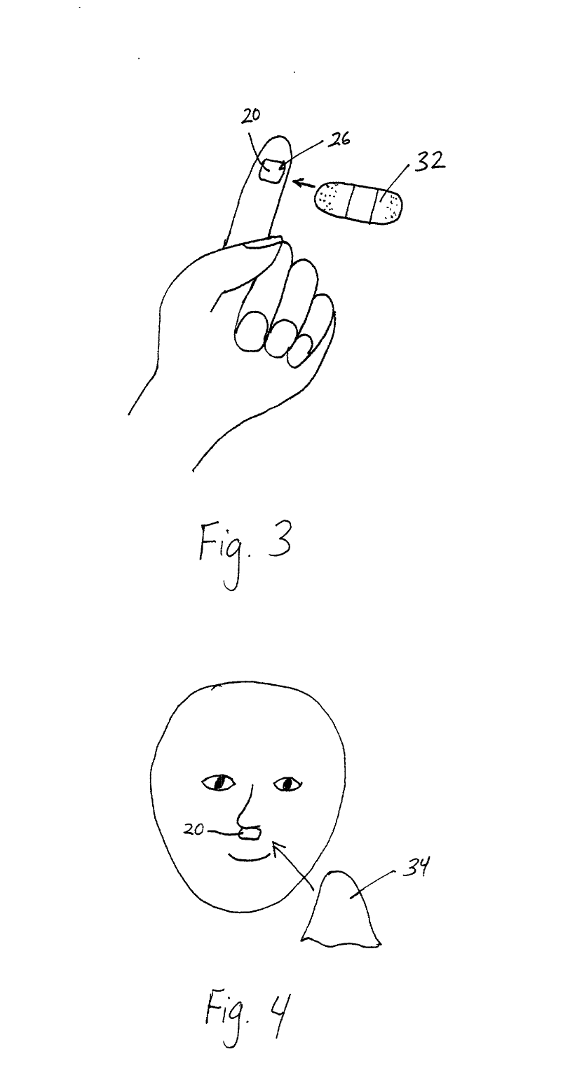

FIG. 2 illustrates an example of the sampling article 20 inside packaging material 30. The packaging material 30 may include any suitable box, bag, pouch, wrapping, padding, or any other materials suitable for maintaining the sampling article 20. Specific types of suitable materials from which the packaging material 30 may be made include paper (cellulose-based) products, polyolefin films and wraps, and hard plastics based on acrylate polymers or polypropylene. The shape and structure of the sampling article 20 may be adapted to fit any potential testing area. For example, the sampling article 20 may be triangular, square, circular, oval, rectangular, octagonal, hexagonal or any other shape designed to provide optimal fit to the body area of interest. The size of the sampling article 20 may vary, depending upon the specific area of the body to which the article 20 will be applied and the type of application for which the article 20 will be used. In general, the substrate 22 may have a maximum length of between about 1 centimeter and about 10 centimeters.

The sampling article 20 is placed directly onto a wearer's skin, as shown in FIG. 3, with the skin-adhering surface 24 in direct contact with the wearer's skin and the skin-emulating surface 26 facing away from the wearer. The sampling article 20 is placed on the wearer in a location and manner representative of actual use conditions. For example, a medicated bandage 32 may be placed over the sampling article 20 shown in FIG. 3 for purposes of sampling a drug transferred from the bandage 32 to the finger, or in this case, to the sampling article 20.

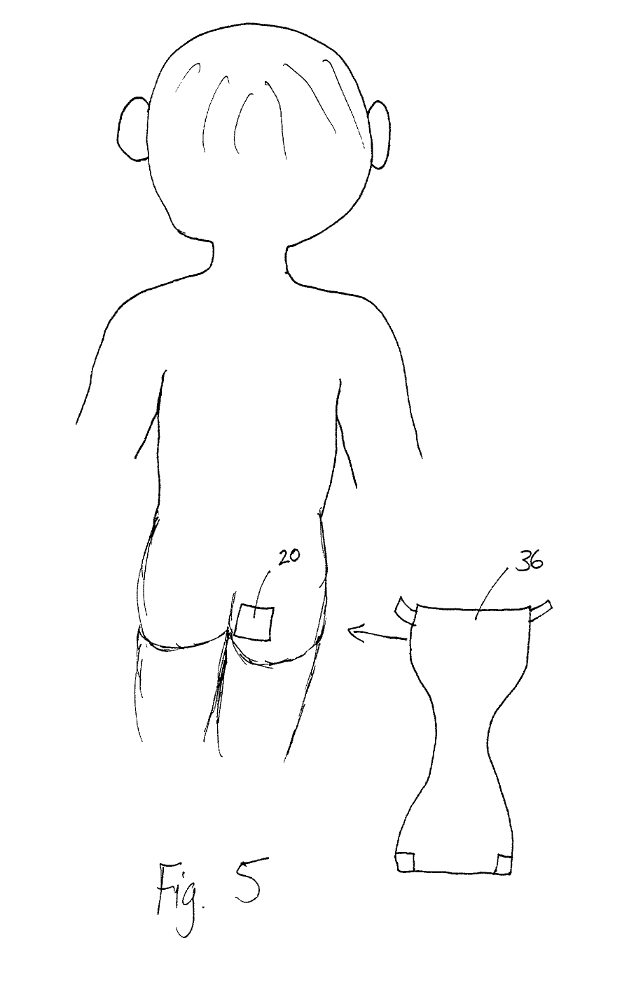

Another example of a suitable location for the sampling article 20 is shown in FIG. 4. In FIG. 4, the sampling article 20 is placed on a wearer's skin just below the wearer's nose. A facial tissue 34 containing a drug, such as an anti-bacterial treatment or an anti-inflammatory treatment, can be used to wipe the wearer's nose in a customary nose-wiping manner, thus transferring the drug to the sampling article 20. The sampling article 20 can then be tested to determine the quantitative and qualitative properties of the drug transferred to a wearer's nasolabial area from the tissue 34 through such nose-wiping action.

Another example of a suitable application of the sampling article 20 is shown in FIG. 5. In FIG. 5, the sampling article 20 is placed on a baby's buttocks, or a portion of the buttocks. A diaper 36 treated with a drug, such as a diaper rash treatment, is then applied to the baby over the sampling article 20. After a predetermined length of time and/or range of motion, the diaper 36 is removed and the sampling article 20 is removed and tested to determine the quantitative and/or qualitative properties of the drug transferred from the diaper 36 to the wearer's buttock region.

By using the non-interfering sampling article 20 of the invention, drug transfer studies may be carried out with unprecedented accuracy. The ability to determine how much of each drug and the types of drugs being transferred to human skin through drug-containing media will enable researchers to assess the effectiveness of such drug-containing media and identify areas where improvements are necessary.

It will be appreciated that details of the foregoing embodiments, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

We claim:

1. An article for measuring external drug transfer to skin surfaces, comprising:
a polyolefin substrate film including at least one of the group consisting of polyethylene and polypropylene, the film having a weight-average molecular weight between about 3 and about 6 kilograms/mole; and
a skin-adhering element attached to the polyolefin substrate film, wherein the article is free of aromatic organic compounds, polyenes, acrylates, esters, waxes, dimethicones and silicone-based compounds.

2. The article of claim 1, wherein the film has a weight-average molecular weight between about 3 and about 4 kilograms/mole.

3. The article of claim 1, wherein the polyolefin substrate film comprises a surface texture having a roughness, calculated as an arithmetic sum of all deviations about a best-fit mean plane through topographical surface data, of between about 19 and about 32 microns.

4. The article of claim 1, wherein the polyolefin substrate film comprises a surface texture having human skin topography.

5. The article of claim 1, wherein the skin-adhering element comprises an adhesive selected from the group consisting of a polyacrylate ester based hydrogel adhesive, a polymethacrylate ester based hydrogel adhesive, a high solids moisture resistant latex pressure-sensitive adhesive, and a polyalkyloxazoline-reinforced acrylic pressure-sensitive adhesive.

6. The article of claim 5, wherein the adhesive comprises components each having a minimum molecular weight of at least 1,500 daltons.

7. The article of claim 5, wherein the adhesive has a boiling point of at least 250° Celsius.

8. The article of claim 1, wherein the skin-adhering element comprises an electrostatic adhesive.

9. The article of claim 8, wherein the polyolefin substrate film has a thickness of less than 80 microns.

10. The article of claim 1, further comprising packaging materials in contact with the polyolefin substrate film, wherein the packaging materials are non-interfering with respect to analysis of the drug being measured.

11. The article of claim 1, wherein the polyolefin substrate film has a shape selected from the group consisting of triangular, square, circular, oval, rectangular, octagonal, and hexagonal.

12. The article of claim 1, wherein the polyolefin substrate film has a maximum length of between about 1 centimeter and about 10 centimeters.

13. An article for measuring external drug transfer to skin surfaces, comprising:
a polytetrafluoroethylene substrate film having a weight-average molecular weight between about 35 and about 65 kilograms/mole; and
a skin-adhering element attached to the polytetrafluoroethylene substrate film, wherein the article is free of aromatic organic compounds, polyenes, acrylates, esters, waxes, dimethicones and silicone-based compounds.

14. The article of claim 13, wherein the film has a weight-average molecular weight between about 45 and about 55 kilograms/mole.

15. The article of claim 13, wherein the polytetrafluoroethylene substrate film comprises a surface texture having a roughness, calculated as an arithmetic sum of all deviations about a best-fit mean plane through topographical surface data, of between about 19 and about 32 microns.

16. The article of claim 13, wherein the polytetrafluoroethylene substrate film comprises a surface texture having human skin topography.

17. The article of claim 13, wherein the skin-adhering element comprises an adhesive selected from the group consisting of a polyacrylate ester based hydrogel adhesive, a polymethacrylate ester based hydrogel adhesive, a high solids moisture resistant latex pressure-sensitive adhesive, and a polyalkyloxazoline-reinforced acrylic pressure-sensitive adhesive.

18. The article of claim 17, wherein the adhesive has a minimum molecular weight of at least 1,500 daltons.

19. The article of claim 17, wherein the adhesive has a boiling point of at least 250° Celsius.

20. The article of claim 13, wherein the skin-adhering element comprises an electrostatic adhesive.

21. The article of claim 20, wherein the polytetrafluoroethylene substrate film has a thickness of less than 80 microns.

22. The article of claim 13, further comprising packaging materials in contact with the polytetrafluoroethylene substrate film, wherein the packaging materials are non-interfering with respect to analysis of the drug being measured.

23. The article of claim 13, wherein the polytetrafluoroethylene substrate film has a shape selected from the group consisting of triangular, square, circular, oval, rectangular, octagonal, and hexagonal.

24. The article of claim 13, wherein the polytetrafluoroethylene substrate film has a maximum length of between about 1 centimeter and about 10 centimeters.

25. An article for measuring external drug transfer to skin surfaces, comprising:
a substrate including at least one of the group consisting of metallic foil film, metallized film, poly(methyl methacrylate), poly(vinyl alcohol), poly(ethylene oxide), poly(ethylene terephthalate), polycaprolactam, poly(hexamethylene adipamide), poly(α-1,6-D-glucose), polydimethylsiloxanes, and poly(cis-1,4-isoprene); and
a skin-adhering element attached to the substrate, wherein the article is free of aromatic organic compounds, polyenes, acrylates, esters, waxes, dimethicones and silicone-based compounds.

26. The article of claim 25, wherein the substrate comprises a surface texture having a roughness, calculated as an arithmetic sum of all deviations about a best-fit mean plane through topographical surface data, of between about 19 and about 32 microns.

27. The article of claim 25, wherein the substrate comprises a surface texture having human skin topography.

28. The article of claim 25, wherein the skin-adhering element comprises an adhesive selected from the group consisting of a polyacrylate ester based hydrogel adhesive, a polymethacrylate ester based hydrogel adhesive, a high solids moisture resistant latex pressure-sensitive adhesive, and a polyalkyloxazoline-reinforced acrylic pressure-sensitive adhesive.

29. The article of claim 28, wherein the adhesive has a minimum molecular weight of at least 1,500 daltons.

30. The article of claim 28, wherein the adhesive has a boiling point of at least 250° Celsius.

31. The article of claim 25, wherein the skin-adhering element comprises an electrostatic adhesive.

32. The article of claim 31, wherein the substrate has a thickness of less than 80 microns.

33. The article of claim 25, further comprising packaging materials in contact with the substrate, wherein the packaging materials are non-interfering with respect to analysis of the drug being measured.

34. The article of claim 25, wherein the substrate has a shape selected from the group consisting of triangular, square, circular, oval, rectangular, octagonal, and hexagonal.

35. The article of claim 25, wherein the substrate has a maximum length of between about 1 centimeter and about 10 centimeters.
